(19) United States
(12) Patent Application Publication
(10) Pub. No.: US 2026/0007794 A1
(43) Pub. Date: Jan. 8, 2026

(54) PROCESS CHALLENGE DEVICE AND METHOD OF MANUFACTURING THEREOF

(71) Applicant: Solventum Intellectual Properties Company, Maplewood, MN (US)

(72) Inventors: Jener de Oliveira, São Paulo (BR); Dorivaldo L. Siqueira, São Paulo (BR); Paul N. Holt, Saint Paul, MN (US)

(73) Assignee: Solventum Intellectual Properties Company, Maplewood, MN (US)

(21) Appl. No.: 19/123,485

(22) PCT Filed: Oct. 17, 2023
(86) PCT No.: PCT/IB2023/060482

§ 371 Date: Apr. 23, 2025

Related U.S. Application Data
(60) Provisional application No. 63/380,816, filed on Oct. 25, 2022.

(51) Int. Cl. A61L 2/28

(52) U.S. Cl. CPC A61L 2/28

(57) ABSTRACT

A process challenge to be used for determining the effectiveness of a sterilization procedure is provided. The process challenge device includes a stack including a plurality of test sheets disposed on top of each other. The process challenge device further includes a test indicator disposed within and enclosed by the plurality of test sheets of the stack. The process challenge device further includes a first flexible sheet including a bottom wall receiving the stack thereon, a plurality of side walls, and a peripheral flange extending from the plurality of side walls. The process challenge device further includes a second flexible sheet disposed on and at least partially engaging with the stack and the peripheral flange. The process challenge device further includes a continuous peripheral seal coupling the second flexible sheet to the peripheral flange. The stack is fully enclosed by the first flexible sheet and the second flexible sheet.

### TECHNICAL FIELD

The present disclosure relates generally to sterilization, and more particularly, relates to a process challenge device to be used for determining the effectiveness of a sterilization procedure. The present disclosure further relates to a method of manufacturing the process challenge device.

### BACKGROUND

Sterilization of medical and hospital equipment may not be effective until a steam sterilant has been in contact with all surfaces of materials being sterilized in a proper combination of time, temperature, and steam quality. In steam sterilizers, such as pre-vacuum steam sterilizers and gravity displacement steam sterilizers, the process of sterilization is conducted in three main phases. In the first phase, air is removed, including air trapped within any porous materials being processed. The first phase is therefore an air removal phase. The second phase is a sterilizing stage, in which a load (i.e., the articles being sterilized) is subjected to steam under pressure for a recognized, predetermined combination of time and temperature to effect sterilization. The third phase is a drying phase in which condensation formed during the first two phases is removed by evacuating the chamber.

Any air that is not removed from the sterilizer during the air removal phase of the cycle or which leaks into the sterilizer during a sub-atmospheric pressure stage due to, for example, faulty gaskets, valves or seals, may form air pockets within any porous materials present. Such air pockets may create a barrier to steam penetration, thereby preventing adequate sterilizing conditions being achieved for all surfaces of the load during the sterilizing phase. For example, these air pockets may prevent the steam from reaching interior layers of materials, such as hospital linens or fabrics. In some other examples, these air pockets may prevent the steam from penetrating hollow spaces of tubes, catheters, syringe needles, and the like. Further, non-condensable gas (generally air) present within the sterilizer is a poor sterilant and may decrease sterilization efficacy. A percentage of non-condensable gas in the steam should be less than or equal to 3.5% by volume. Therefore, the presence of air pockets and/or non-condensable gas may affect a steam quality of the steam sterilant. As a result, proper sterilization may not occur due to reduced steam quality. A few more factors that may affect steam quality include insufficient steam supply, water quality, degassing, design of the sterilizer chamber, etc.

It can be stated that proper sterilization may not occur due to inappropriate steam quality, air removal, time and temperature of sterilization. For monitoring whether the sterilization process is being conducted at adequate temperatures, with adequate steam quality, air removal, and for adequate time period, process challenge devices and/or Bowie-Dick test devices are used. Process challenge devices may be used to evaluate steam parameters, such as steam quality, temperature, and time of sterilization procedure. Bowie-Dick test devices are more focused on monitoring air removal inside the sterilizer chamber.

### SUMMARY

In a first aspect, the present disclosure provides a process challenge device to be used for determining the effectiveness of a sterilization procedure. The process challenge device includes a stack including a plurality of test sheets disposed on top of each other. The stack further includes an outer surface. The process challenge device further includes a test indicator disposed within and enclosed by the plurality of test sheets of the stack. The process challenge device further includes a first flexible sheet at least partially conforming to the outer surface of the stack. The first flexible sheet includes a bottom wall receiving the stack thereon, a plurality of side walls extending from the bottom wall, and a peripheral flange extending from the plurality of side walls and substantially parallel to the bottom wall. The bottom wall and the plurality of side walls of the first flexible sheet together define a sheet cavity therebetween. The sheet cavity receives the stack therein and is dimensioned such that each of the plurality of side walls at least partially engages with the stack. An inclination angle between each of the plurality of side walls and the bottom wall is from 80 degrees to 100 degrees. The process challenge device further includes a second flexible sheet disposed on and at least partially engaging with the stack and the peripheral flange of the first flexible sheet. The second flexible sheet covers the stack. The process challenge device further includes a continuous peripheral seal coupling the second flexible sheet to the peripheral flange, such that the stack is fully enclosed by the first flexible sheet and the second flexible sheet.

In a second aspect, the present disclosure provides a method of manufacturing a process challenge device to be used for determining the effectiveness of a sterilization procedure. The method includes providing a stack including a plurality of test sheets disposed on top of each other. The stack further includes an outer surface. The method further includes placing a test indicator within the plurality of test sheets of the stack. The method further includes placing the stack on a first flexible sheet. The method further includes placing the first flexible sheet along with the stack within a crib including a crib bottom wall, a plurality of crib side walls extending from the crib bottom wall, a crib peripheral flange extending from the plurality of crib side walls and substantially parallel to the crib bottom wall, and a crib cavity defined between the crib bottom wall and the plurality of crib side walls. The crib cavity at least partially receives the first flexible sheet along with the stack therein. Placement of the first flexible sheet along with the stack within the crib deforms the first flexible sheet to at least partially conform to the outer surface of the stack. Deformation of the first flexible sheet forms a bottom wall at least partially engaging with the crib bottom wall, a plurality of side walls extending from the bottom wall and at least partially engaging with the corresponding plurality of crib side walls, and a peripheral flange extending from the plurality of side walls and substantially parallel to the bottom wall. The peripheral flange of the first flexible sheet at least partially engages with the crib peripheral flange. The crib cavity is dimensioned such that each of the plurality of side walls of the first flexible sheet at least partially engages with the stack. Further, the crib cavity is dimensioned such that an inclination angle between each of the plurality of side walls and the bottom wall of the first flexible sheet is from 80 degrees to 100 degrees. The method further includes placing a second flexible sheet on the stack and the first flexible sheet, such that the second flexible sheet covers the stack and at least partially engages with the stack and the peripheral flange of the first flexible sheet. The method further includes forming a continuous peripheral seal between the second flexible sheet and the peripheral flange, thereby coupling the second flexible sheet to the peripheral flange and fully enclosing the stack between the first flexible sheet and the second flexible sheet. The method further includes removing the first flexible sheet along with the stack from the crib.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a perspective view of a process challenge device to be used for determining the effectiveness of a sterilization procedure, according to an embodiment of the present disclosure;

FIG. 2 is an exploded view of a stack, a test indicator, and a first flexible sheet of the process challenge device of FIG. 1, according to an embodiment of the present disclosure;

FIG. 3 is an exploded view of the stack and the first flexible sheet of the process challenge device of FIG. 1, and a crib, according to an embodiment of the present disclosure;

FIG. 4 is a perspective view of the stack and the first flexible sheet of the process challenge device of FIG. 1, and the crib, wherein the first flexible sheet along with the stack is placed within the crib, according to an embodiment of the present disclosure;

FIG. 5 is a perspective view of the first flexible sheet after being placed within the crib, according to an embodiment of the present disclosure;

FIG. 6 is an perspective view of a combination of the stack, the first flexible sheet and the crib of FIG. 4, and a second flexible sheet of the process challenge device of FIG. 1 being placed on the stack and the first flexible sheet, according to an embodiment of the present disclosure;

FIG. 7 is a perspective view of a combination of the first flexible sheet, the second flexible sheet and the crib of FIG. 6, and a sealing plate being placed on the second flexible sheet, according to an embodiment of the present disclosure;

FIG. 8 is a perspective view of the first flexible sheet, the second flexible sheet, the crib, and the scaling plate of FIG. 7, wherein the first flexible sheet and the second flexible sheet are shown as being at least partially received between the crib and the sealing plate, according to an embodiment of the present disclosure;

FIG. 9 is a perspective view of the process challenge device of FIG. 1, the crib of FIG. 3, and the sealing plate of FIG. 7, wherein the crib and the sealing plate are shown as being removed from the process challenge device, according to an embodiment of the present disclosure;

FIG. 10 is a perspective view of a crib, according to another embodiment of the present disclosure; FIG. 11 is a perspective view of a crib, according to yet another embodiment of the present disclosure; and

FIG. 12 is a flow chart for a method of manufacturing the process challenge device of FIG. 1, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

As used herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

As used herein, the term “sheet” generally refers to a material with a very high ratio of length or width to thickness. A sheet has two major surfaces defined by a length and width. Sheets typically have good flexibility and can be used for a wide variety of applications, including displays. Sheets may also be of thickness or material composition, such that they are semi-rigid or rigid. Sheets described in the present disclosure may be composed of various polymeric materials. Sheets may be monolayer, multilayer, or blend of different polymers.

The term “coupled”, or “connected” may include direct physical connections between two or more components, or indirect physical connections between two or more components that are connected together by one or more additional components. For example, a first component may be coupled to a second component by being directly connected together or by being connected by a third component.

The term “non-thermoformable” refers to a sheet that is not capable of being formed or thermoformed into a desired shape by the application of a differential pressure between the sheet and a mold, by the application of heat, by the combination of the application of heat and a differential pressure between the sheet and a mold, or by any thermoforming technique known to those skilled in the art.

The term “non-moldable” may mean that a member is not easily plastically deformable when in use, in contrast to a moldable member.

Steam sterilizers are widely used in medical centers and hospitals to sterilize medical equipment. Frequent testing or monitoring of steam quality may be essential to ensure a safe use of the medical equipment in a medical treatment. In other words, regular testing may have to be conducted to check effectiveness of air removal during air removal phase of the sterilization process, prior to subjecting the steam to a given load (i.e., medical equipment). One of the ways to monitor steam quality of the steam sterilant is a Bowie-Dick test. In general, the Bowie-Dick test uses an indicator disposed between a plurality of paper sheets so as to form a test pack. In some cases, the indicator is a chemical indicator. In some cases, the indicator is a bio indicator. In some cases, the test pack used in the Bowie-Dick test includes a disposable test pack.

Generally, the test pack is packaged or placed inside a wrap, such that the package may represent the resistance (i.e., resistance to steam sterilant) of various flow channels leading to hidden spaces of tubes, catheters, syringe needles, and the like. Conventionally, the test pack is packaged inside the wrap by manually folding various portions of the wrap. In other words, a user has to manually fold the wrap multiple times to completely pack the plurality of paper sheets and the indicator. Such a process may be tedious and time consuming for the user. Moreover, such a process may also fail to provide a precise wrapping of the test pack. In some applications, machinery may be used to avoid manual folding. However, such machinery may add cost and complexity to the overall folding process. There may also be a possibility of the wrap coming apart or unfolding during handling of the wrap.

The present disclosure relates to a process challenge device to be used for determining the effectiveness of a sterilization procedure. The process challenge device includes a stack including a plurality of test sheets disposed on top of each other. The stack further includes an outer surface. The process challenge device further includes a test indicator disposed within and enclosed by the plurality of test sheets of the stack. The process challenge device further includes a first flexible sheet at least partially conforming to the outer surface of the stack. The first flexible sheet includes a bottom wall receiving the stack thereon, a plurality of side walls extending from the bottom wall, and a peripheral flange extending from the plurality of side walls and substantially parallel to the bottom wall. The bottom wall and the plurality of side walls of the first flexible sheet together define a sheet cavity therebetween. The sheet cavity receives the stack therein and is dimensioned such that each of the plurality of side walls at least partially engages with the stack. An inclination angle between each of the plurality of side walls and the bottom wall is from 80 degrees to 100 degrees. The process challenge device further includes a second flexible sheet disposed on and at least partially engaging with the stack and the peripheral flange of the first flexible sheet. The second flexible sheet covers the stack. The process challenge device further includes a continuous peripheral seal coupling the second flexible sheet to the peripheral flange, such that the stack is fully enclosed by the first flexible sheet and the second flexible sheet.

By coupling the second flexible sheet to the peripheral flange of the first flexible sheet through the continuous peripheral seal, the stack including the test indicator is firmly packed within the second flexible sheet and the first flexible sheet. Further, the continuous peripheral seal assists in the packing of the stack within the second flexible sheet and the first flexible sheet without any need of manual folding of the second flexible sheet and the first flexible sheet.

As each of the plurality of side walls at least partially engages with the stack and the inclination angle between each of the plurality of side walls and the bottom wall is from 80 degrees to 100 degrees, a desirable packing density of the stack may be achieved. In some embodiments, the stack includes a packing density from 0.55 g/cm³ to 0.75 g/cm³. Such a packing density of the stack may keep the stack including the test indicator intact within the second flexible sheet and the first flexible sheet, which may eventually improve the accuracy of Bowie-Dick test results. As a result, the process challenge device may precisely determine the effectiveness of the sterilization procedure.

The present disclosure further relates to a method of manufacturing a process challenge device to be used for determining the effectiveness of a sterilization procedure. The method includes providing a stack including a plurality of test sheets disposed on top of each other. The stack further includes an outer surface. The method further includes placing a test indicator within the plurality of test sheets of the stack. The method further includes placing the stack on a first flexible sheet. The method further includes placing the first flexible sheet along with the stack within a crib including a crib bottom wall, a plurality of crib side walls extending from the crib bottom wall, a crib peripheral flange extending from the plurality of crib side walls and substantially parallel to the crib bottom wall, and a crib cavity defined between the crib bottom wall and the plurality of crib side walls. The crib cavity at least partially receives the first flexible sheet along with the stack therein. Placement of the first flexible sheet along with the stack within the crib deforms the first flexible sheet to at least partially conform to the outer shape of the stack. Deformation of the first flexible sheet forms a bottom wall at least partially engaging with the crib bottom wall, a plurality of side walls extending from the bottom wall and at least partially engaging with the corresponding plurality of crib side walls, and a peripheral flange extending from the plurality of side walls and substantially parallel to the bottom wall. The peripheral flange of the first flexible sheet at least partially engages with the crib peripheral flange. The crib cavity is dimensioned such that each of the plurality of side walls of the first flexible sheet at least partially engages with the stack. Further, the crib cavity is dimensioned such that an inclination angle between each of the plurality of side walls and the bottom wall of the first flexible sheet is from 80 degrees to 100 degrees. The method further includes placing a second flexible sheet on the stack and the first flexible sheet, such that the second flexible sheet covers the stack and at least partially engages with the stack and the peripheral flange of the first flexible sheet. The method further includes forming a continuous peripheral seal between the second flexible sheet and the peripheral flange, thereby coupling the second flexible sheet to the peripheral flange and fully enclosing the stack between the first flexible sheet and the second flexible sheet. The method further includes removing the first flexible sheet along with the stack from the crib.

The crib is designed such that the inclination angle between each of the plurality of side walls and the bottom wall of the first flexible sheet is from 80 degrees to 100 degrees. Moreover, due to placement of the first flexible sheet along with the stack within the crib, each of the plurality of side walls of the first flexible sheet at least partially engages with the stack. This may provide the stack with a desirable packing density (e.g., the packing density from 0.55 g/cm³ to 0.75 g/cm³). As a result, the proposed method of manufacturing may keep the stack including the test indicator intact within the second flexible sheet and the first flexible sheet, which may eventually improve the accuracy of Bowie-Dick test results.

Further, the proposed method of manufacturing the process challenge device does not involve any manual steps of folding the first flexible sheet and/or the second flexible sheet. Therefore, while manufacturing the process challenge device by the proposed method, any error related to manual folding of sheets or wraps is avoided. Moreover, the proposed method of manufacturing the process challenge device may be easy to perform and may be cost effective as compared to conventional techniques and methods of manufacturing the process challenge devices.

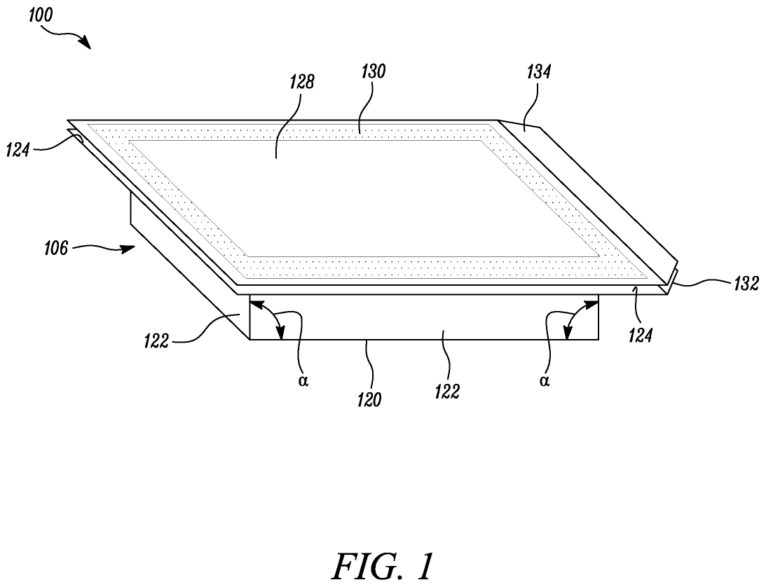

Referring now to Figures, FIG. 1 is a perspective view of a process challenge device 100 to be used for determining the effectiveness of a sterilization procedure, according to an embodiment of the present disclosure. The process challenge device 100 is used to conduct Bowie-Dick tests and offer a resistance to a steam sterilant that is substantially same as the resistance of various flow channels leading to hidden spaces of tubes, catheters, syringe needles, and the like.

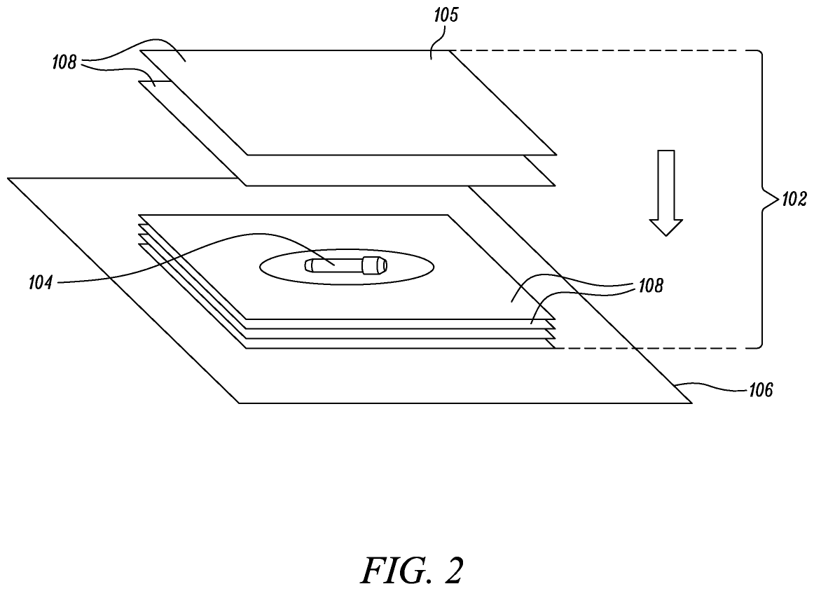

The process challenge device 100 includes a stack 102 (shown in FIG. 2), a test indicator 104, and a first flexible sheet 106. FIG. 2 is an exploded view of the stack 102, the test indicator 104, and the first flexible sheet 106 of the process challenge device 100 of FIG. 1, according to an embodiment of the present disclosure. In FIG. 2, the first flexible sheet 106 is shown in an undeformed state for illustrative purposes. However, in the process challenge device 100 of FIG. 1, the first flexible sheet 106 is in a deformed state. In the illustrated embodiment, the first flexible sheet 106 has a rectangular shape in the undeformed sate.

Referring to FIGS. 1 and 2, the stack 102 includes a plurality of test sheets 108 disposed on top of each other. In some embodiments, each of the plurality of test sheets 108 is made of paper, or paper and polyurethane foam. The presence of polyurethane foam in the paper may help to keep the plurality of test sheets 108 with a desirable pressure. Each of the plurality of test sheets 108 may be made of porous paper. Therefore, the plurality of test sheets 108 are permeable to the steam sterilant used in the sterilization procedure and a gas (e.g., ethylene oxide). The stack 102 further includes an outer surface 105. The outer surface 108 of the stack 102 is defined by a pair of outer (i.e., top and bottom) test sheets 108 of the stack 102 and a combined thickness of the plurality of test sheets 108 when stacked on top of each other. In the illustrated embodiment, the outer surface 108 has a substantially cuboidal shape as the test sheets 108 are rectangular.

The test indicator 104 is disposed within and enclosed by the plurality of test sheets 108 of the stack 102. At least some of the plurality of test sheets 108 adjacent to the test indicator 104 may deform to enclose the test indicator 104 within the stack 102. The test indicator 104 may be a biological indicator or a chemical indicator. In some embodiments, there may be two or more test indicators 104 disposed within and enclosed by the plurality of test sheets 108. The test indicator 104 may be chosen to be used with sterilization conditions to be employed in a particular sterilization process. Moreover, the test indicator 104 can be chosen based upon the amount of exposure to sterilization conditions required to cause the test indicator 104 to indicate that the exposure has occurred. The choice of the test indicator 104 can thereby be used to increase or decrease the resistance of the process challenge device 100. For manufacturing the process challenge device 100, the test indicator 104 is placed within the plurality of test sheets 108 of the stack 102, as shown in FIG. 2.

In the process challenge device 100 (shown in FIG. 1), the first flexible sheet 106 at least partially conforms to the outer surface 105 of the stack 102. For manufacturing the process challenge device 100. once the test indicator 104 is placed within the plurality of test sheets 108 of the stack 102, the stack 102 is placed on the first flexible sheet 106 (in the undeformed state), as shown in FIG. 2.

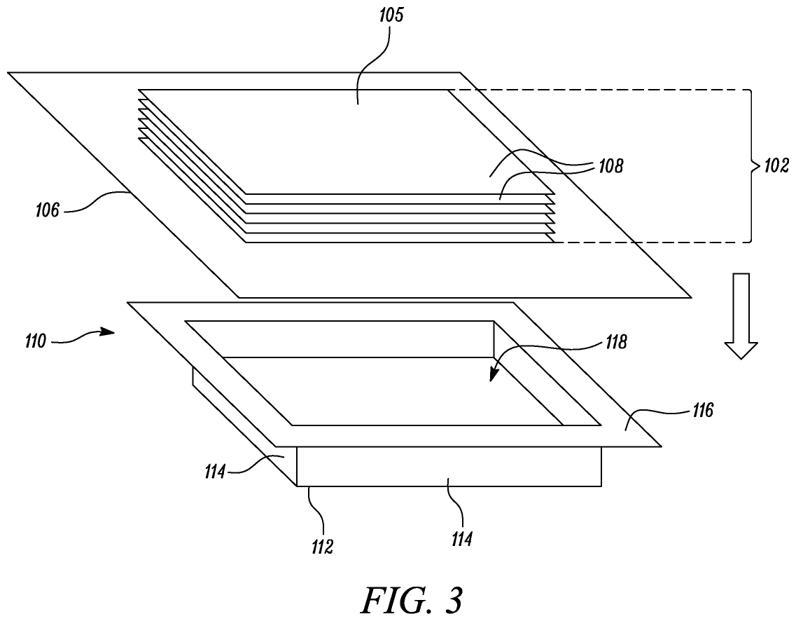

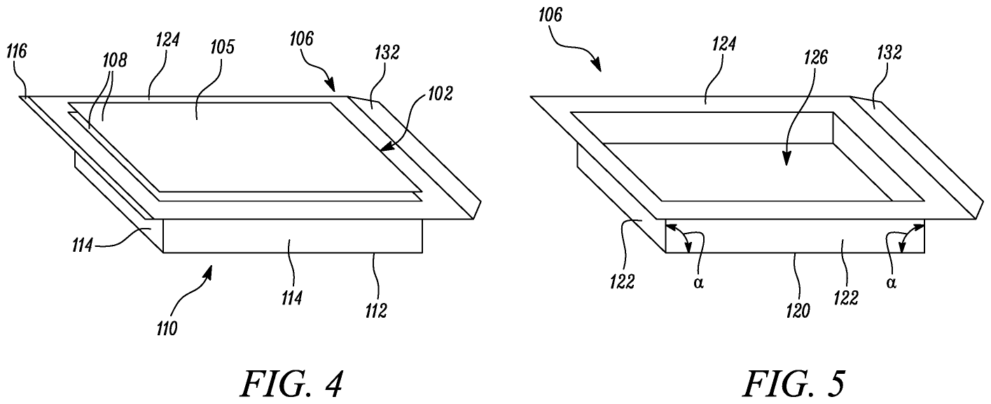

FIG. 3 is an exploded view of the stack 102 and the first flexible sheet 106 of the process challenge device 100 of FIG. 1, and a crib 110, according to an embodiment of the present disclosure. The crib 110 may be a rigid metallic member. For manufacturing the process challenge device 100, once the stack 102 is placed on the first flexible sheet 106 (in undeformed state, shown in FIG. 2), the first flexible sheet 106 along with the stack 102 is placed within the crib 110. FIG. 4 is a perspective view of the stack 102 and the first flexible sheet 106 of the process challenge device of FIG. 1, and the crib 110, wherein the first flexible sheet 106 along with the stack 102 is placed within the crib 110, according to an embodiment of the present disclosure.

Referring to FIGS. 3 and 4, the crib 110 includes a crib bottom wall 112, a plurality of crib side walls 114 extending from the crib bottom wall 112, and a crib peripheral flange 116 extending from the plurality of crib side walls 114 and substantially parallel to the crib bottom wall 112. The crib 110 further includes a crib cavity 118 defined between the crib bottom wall 112 and the plurality of crib side walls 114. In the illustrated embodiment, the crib bottom wall 112 and the plurality of crib side walls 114 are rectangular. A number of the crib side walls 114 is four. Further, the crib cavity 118 has a cuboidal shape. Moreover, the crib peripheral flange 116 has rectangular inner and outer edges.

Upon placement of the first flexible sheet 106 along with the stack 102 within the crib 110, the crib cavity 118 at least partially receives the first flexible sheet 106 along with the stack 102 therein. Moreover, placement of the first flexible sheet 106 along with the stack 102 within the crib 110 deforms the first flexible sheet 106 to at least partially conform to the outer surface 105 of the stack 102. FIG. 5 is a perspective view of the first flexible sheet 106 after being placed within the crib 110, according to an embodiment of the present disclosure. In other words. FIG. 5 is a perspective view of the first flexible sheet 106 when it is in the deformed state.

Referring to FIGS. 3 to 5, after the first flexible sheet 106 is deformed, the first flexible sheet 106 includes a bottom wall 120 (shown in FIG. 5) receiving the stack 102 thereon, a plurality of side walls 122 extending from the bottom wall 120, and a peripheral flange 124 extending from the plurality of side walls 122 and substantially parallel to the bottom wall 120. In other words, after deformation of the first flexible sheet 106, the bottom wall 120 of the first flexible sheet 106 is formed which at least partially engages with the crib bottom wall 112. Further, after deformation of the first flexible sheet 106, the plurality of side walls 122 of the first flexible sheet 106 are formed which at least partially engage with the corresponding plurality of crib side walls 114. Further, after deformation of the first flexible sheet 106, the peripheral flange 124 of the first flexible sheet 106 is formed which least partially engages with the crib peripheral flange 116.

The bottom wall 120 and the plurality of side walls 122 of the first flexible sheet 106 together define a sheet cavity 126 therebetween. The sheet cavity 126 receives the stack 102 therein and is dimensioned such that each of the plurality of side walls 122 at least partially engages with the stack 102. Specifically, the crib cavity 118 is dimensioned such that each of the plurality of side walls 122 of the first flexible sheet 106 at least partially engages with the stack 102. Further, the crib cavity 118 is dimensioned such that an inclination angle α between each of the plurality of side walls 122 and the bottom wall 120 of the first flexible sheet 106 is from 80 degrees to 100 degrees. Such values of the inclination angle α between each of the plurality of side walls 122 and the bottom wall 120 of the first flexible sheet 106 may help the stack 102 to obtain a desirable packing density, which will be discussed later in the description. In some embodiments, the inclination angle α may be about 90 degrees.

In the illustrated embodiment, the bottom wall 120 and the plurality of side walls 122 of the first flexible sheet 106 are rectangular. A number of the side walls 122 is four. Further, the sheet cavity 126 has a cuboidal shape. Moreover, the peripheral flange 124 has rectangular inner and outer edges.

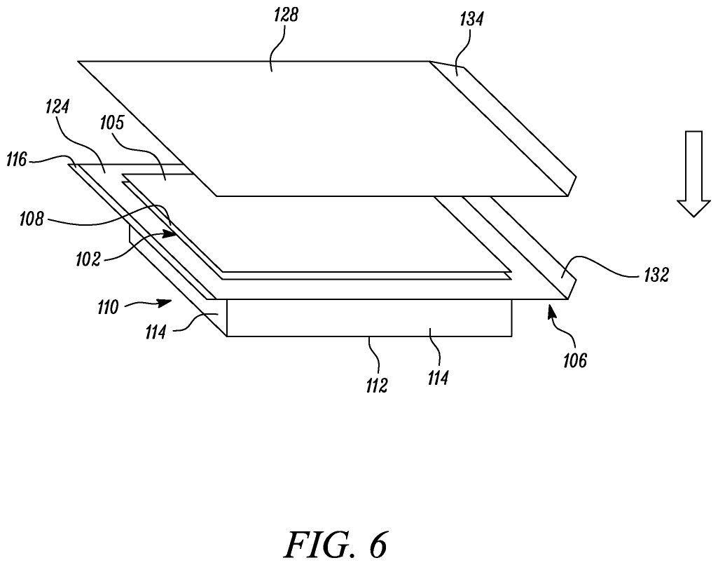

Referring again to FIG. 1, the process challenge device 100 further includes a second flexible sheet 128 disposed on and at least partially engaging with the stack 102 (shown in FIG. 3) and the peripheral flange 124 of the first flexible sheet 106. The second flexible sheet 128 covers the stack 102. In the illustrated embodiment, the second flexible sheet 128 is rectangular. FIG. 6 is a perspective view of a combination of the stack 102, the first flexible sheet 106 and the crib 110 of FIG. 4, and the second flexible sheet 128 of the process challenge device 100 of FIG. 1 being placed on the stack 102 and the first flexible sheet 106, according to an embodiment of the present disclosure.

For manufacturing the process challenge device 100, once the first flexible sheet 106 along with the stack 102 is placed within the crib 110, the second flexible sheet 128 is placed on the stack 102 and the first flexible sheet 106, such that the second flexible sheet 128 covers the stack 102 and at least partially engages with the stack 102 and the peripheral flange 124 of the first flexible sheet 106.

In some embodiments, each of the first flexible sheet 106 and the second flexible sheet 128 is devoid of any openings having an area greater than or equal to 0.5 mm². This may prevent any unrestricted and non-uniform flow of steam sterilant through the first flexible sheet 106 and the second flexible sheet 128. Any unrestricted and non-uniform flow of steam sterilant may compromise any test results provided by the process challenge device 100. In some embodiments, each of the first flexible sheet 106 and the second flexible sheet 128 is non-thermoformable and non-moldable. Therefore, each of the first flexible sheet 106 and the second flexible sheet 128 may not be capable of being formed or thermoformed into a desired shape by the application of a differential pressure between the corresponding flexible sheet and a mold, by the application of heat, by the combination of the application of heat and a differential pressure between the corresponding flexible sheet and a mold, or by any thermoforming technique known to those skilled in the art. Further, each of the first flexible sheet 106 and the second flexible sheet 128 may not be easily plastically deformable when in use. In some embodiments, each of the first flexible sheet 106 and the second flexible sheet 128 is permeable to the steam sterilant used in the sterilization procedure and a gas, such as ethylene oxide.

In some embodiments, each of the first flexible sheet 106 and the second flexible sheet 128 is made from a nonwoven material. The nonwoven material means a fabric or a web having a structure of individual fibers or threads which are interlaid, but not in an identifiable manner as in a knitted fabric. Nonwoven fabrics or webs have been formed from many processes, such as, for example, meltblowing processes, spunbonding processes, and bonded carded web processes. In some embodiments, the nonwoven material includes a three-layered spunbond-meltblown-spunbond (SMS) construction. In the SMS construction of the nonwoven material, outermost layers of the SMS construction provide mechanical protection to the internal content and the middle layer is primarily responsible for microbial filtration. In some embodiments, the nonwoven material includes at least a portion of polyolefin fibers (i.e., polypropylene or polyethylene). The permeability of the nonwoven material may range from about 15 to about 500 cubic feet per minute (CFM).

Referring again to FIG. 1, the process challenge device 100 further includes a continuous peripheral seal 130 coupling the second flexible sheet 128 to the peripheral flange 124 of the first flexible sheet 106, such that the stack 102 (shown in FIG. 3) is fully enclosed by the first flexible sheet 106 and the second flexible sheet 128. In some embodiments, the continuous peripheral seal 130 is a heat seal. The second flexible sheet 128 and the peripheral flange 124 of the first flexible sheet 106 may be heat sealed together by using a heat-scaling device (e.g., a heat sealer). In some embodiments, the continuous peripheral seal 130 is an ultrasonic seal. The second flexible sheet 128 and the peripheral flange 124 of the first flexible sheet 106 may be ultrasonically sealed together by using an ultrasonic sealing device (e.g., an ultrasonic scaler). The continuous peripheral seal 130 assists in desirable packing of the stack 102 within the second flexible sheet 128 and the first flexible sheet 106 without any need of manual folding of the second flexible sheet 128 and the first flexible sheet 106.

In some embodiments, for forming the continuous peripheral seal 130 between the second flexible sheet 128 and the peripheral flange 124 of the first flexible sheet 106, each of the first flexible sheet 106 and the second flexible sheet 128 has a melting temperature (scaling temperature) greater than 130° C, and less than 220° C. However, in some embodiments, each of the first flexible sheet 106 and the second flexible sheet 128 may also have a melting temperature of about 110° C. or about 240° C.

After forming the continuous peripheral seal 130 to couple the second flexible sheet 128 to the peripheral flange 124 of the first flexible sheet 106, the stack 102 includes a packing density from 0.55 g/cm³ to 0.75 g/cm³. In some embodiments, the stack 102 includes the packing density of about 0.63 g/cm³.

Such a packing density of the stack 102 may keep the stack 102 including the test indicator 104 intact within the second flexible sheet 128 and the first flexible sheet 106, which may eventually improve the accuracy of Bowie-Dick test results. As a result, the process challenge device 100 may precisely determine the effectiveness of the sterilization procedure.

In some embodiments, the first flexible sheet 106 further includes a first tab 132 disposed adjacent to the continuous peripheral seal 130 and unsealed to the second flexible sheet 128. The first tab 132 is formed by bending one of the edges of the first flexible sheet 106 prior to forming the continuous peripheral seal 130. The second flexible sheet 128 includes a second tab 134 disposed adjacent to the continuous peripheral seal 130 and unsealed to the first flexible sheet 106. The second tab 134 is formed by bending the corresponding edge of the second flexible sheet 128 prior to forming the continuous peripheral seal 130. Further, the first tab 132 and the second tab 134 are unscaled from each other.

After the sterilization procedure ends, an operator may pull the first tab 132 and the second tab 134 to unseal the first flexible sheet 106 and the second flexible sheet 128 and thereby access the test indicator 104 to evaluate the result of Bowie-Dick test. Therefore, the inclusion of the first tab 132 and the second tab 134 may case a process of opening the process challenge device 100.

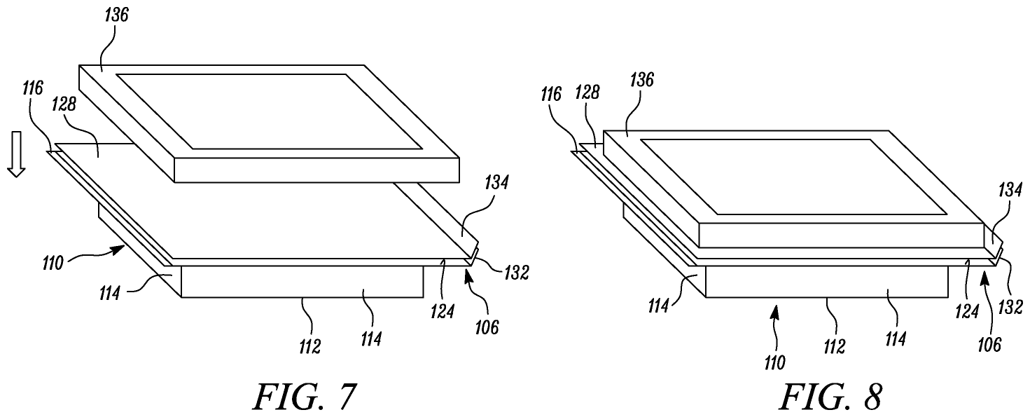

FIG. 7 is a perspective view of a combination of the first flexible sheet 106, the second flexible sheet 128 and the crib 110 of FIG. 6, and a sealing plate 136 being placed on the second flexible sheet 128, according to an embodiment of the present disclosure. In some embodiments, the continuous peripheral seal 130 (shown in FIG. 1) is formed by using the sealing plate 136. Specifically, the second flexible sheet 128 and the peripheral flange 124 of the first flexible sheet 106 are at least partially received between the scaling plate 136 and the crib peripheral flange 116. FIG. 8 is a perspective view of the first flexible sheet 106, the second flexible sheet 128. the crib 110, and the sealing plate 136 of FIG. 7, wherein the first flexible sheet 106 and the second flexible sheet 128 are shown as at least partially received between the crib 110 and the scaling plate 136. The sealing plate 136 is pressed on the second flexible sheet 128 and the peripheral flange 124 to form the continuous peripheral seal 130 and thereby couple the first flexible sheet 106 to the second flexible sheet 128.

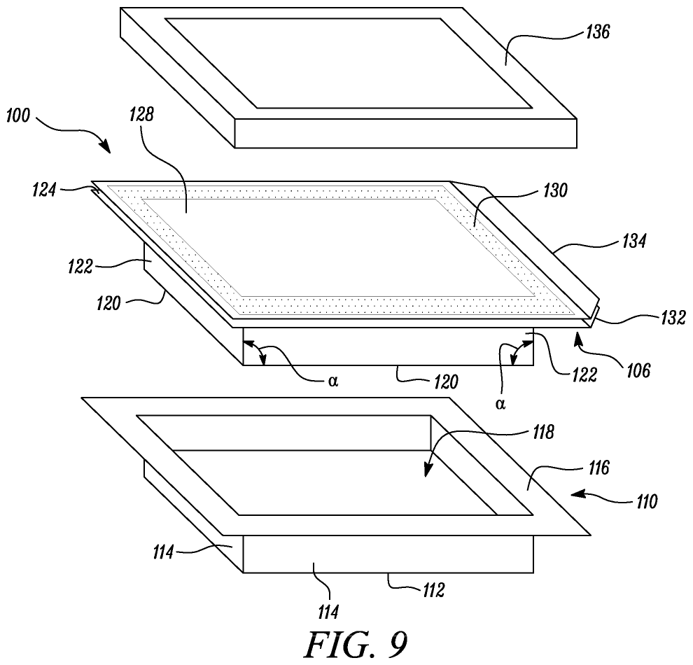

After the continuous peripheral seal 130 is formed, the sealing plate 136 and the crib 110 are removed. FIG. 9 is a perspective view of the process challenge device 100, the crib 110, and the scaling plate 136, wherein the crib 110 and the scaling plate 136 are shown as being removed from the process challenge device 100, according to an embodiment of the present disclosure.

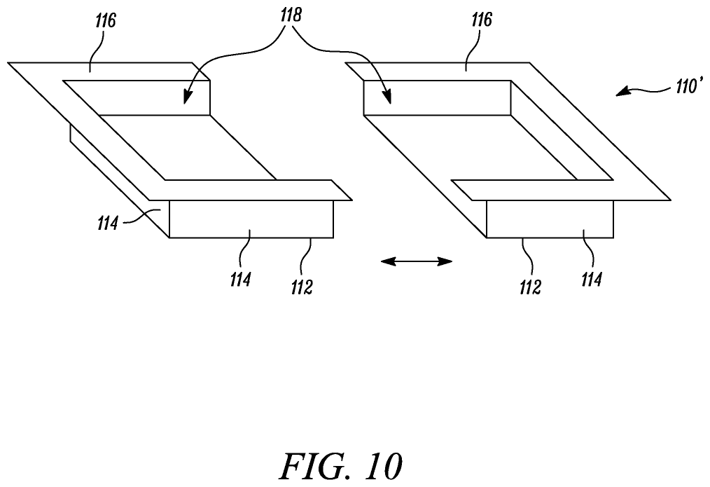

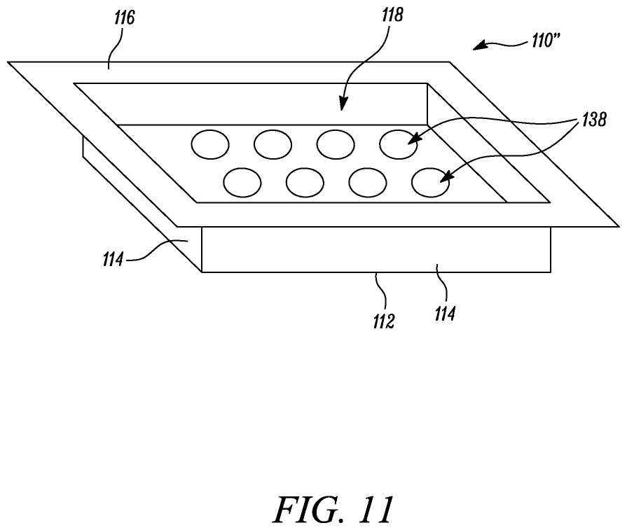

FIG. 10 is a perspective view of a crib 110′, according to an embodiment of the present disclosure. The crib 110′ is substantially similar and functionally equivalent to the crib 110 shown in FIG. 3, with common components being referred to by same numerals. However, the crib 110′ has a two-piece construction (instead of the single piece construction of the crib 110). The two-piece construction of the crib 110′ may allow an easy allowance of the stack 102 and the first flexible sheet 106 within the crib cavity 118. In other words, the two-piece construction of the crib 110′ may facilitate insertion of the first flexible sheet 106 along with the stack 102 within the crib 110′. Further, the two-piece construction of the crib 110′ may facilitate removal of the crib 110′ from the process challenge device 100.

FIG. 11 is a perspective view of a crib 110″, according to an embodiment of the present disclosure. The crib 110″ is substantially similar and functionally equivalent to the crib 110 shown in FIG. 3, with common components being referred to by same numerals. However, the crib 110″ includes a plurality of vacuum channels 138 located therein in order to position the first flexible sheet 106 (shown in FIG. 3) in the crib cavity 118. The vacuum channels 138 create vacuum inside the crib cavity 118 and thereby improve positioning of the first flexible sheet 106 and the stack 102 within the crib 110″

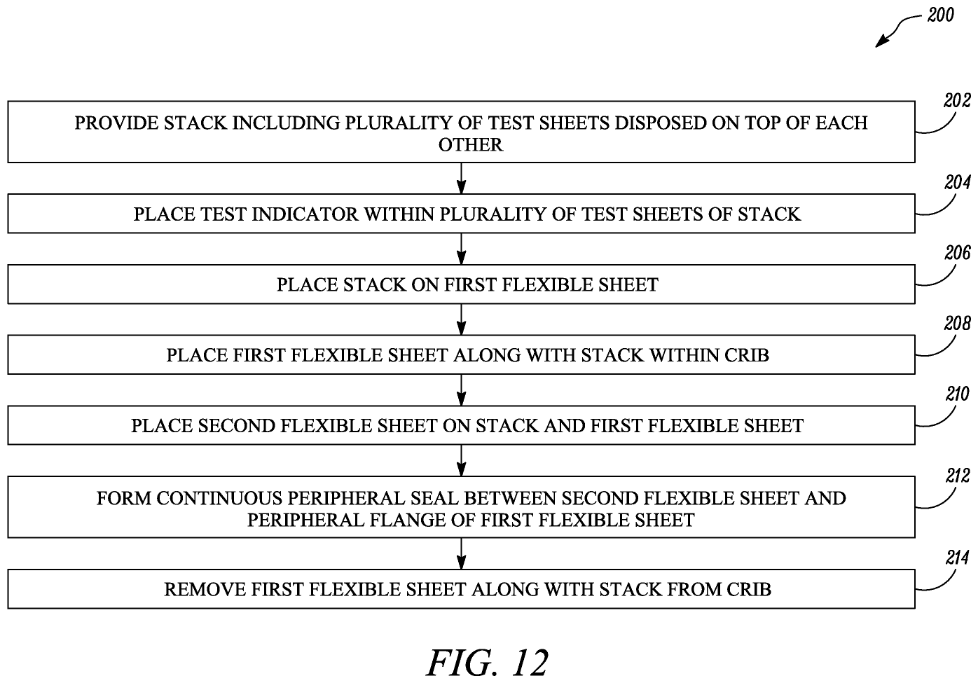

FIG. 12 is flow chart for a method 200 of manufacturing the process challenge device 100 of FIG. 1, according to an embodiment of the present disclosure. Referring to FIGS. 2 to 12, at step 202, the method 200 includes providing the stack 102 (shown in FIGS. 2 and 3) including the plurality of test sheets 108 disposed on top of each other. At step 204, the method 200 includes placing the test indicator 104 (shown in FIG. 2) within the plurality of test sheets 108 of the stack 102. At step 206, the method 200 includes placing the stack 102 on the first flexible sheet 106 (shown in FIG. 3).

At step 208, the method 200 includes placing the first flexible sheet 106 along with the stack 102 within the crib 110 (shown in FIGS. 3 and 4). Placement of the first flexible sheet 106 along with the stack 102 within the crib 110 deforms the first flexible sheet 106 to at least partially conform to the outer surface 105 of the stack 102. Further, deformation of the first flexible sheet 106 (shown in FIG. 5) forms the bottom wall 120 at least partially engaging with the crib bottom wall 112, the plurality of side walls 122 extending from the bottom wall 120 and at least partially engaging with the corresponding plurality of crib side walls 114, and the peripheral flange 124 extending from the plurality of side walls 122 and substantially parallel to the bottom wall 120.

At step 210, the method 200 includes placing the second flexible sheet 128 (shown in FIGS. 6 and 7) on the stack 102 and the first flexible sheet 106, such that the second flexible sheet 128 covers the stack 102 and at least partially engages with the stack 102 and the peripheral flange 124 of the first flexible sheet 106.

At step 212, the method 200 includes forming the continuous peripheral seal 130 (shown in FIGS. 1 and 9) between the second flexible sheet 128 and the peripheral flange 124, thereby coupling the second flexible sheet 128 to the peripheral flange 124 and fully enclosing the stack 102 between the first flexible sheet 106 and the second flexible sheet 128. In some embodiments, the continuous peripheral seal 130 is formed by heat sealing. In some embodiments, the continuous peripheral seal 130 is formed by ultrasonic scaling. In some embodiments, forming the continuous peripheral seal 130 includes at least partially receiving the second flexible sheet 128 and the peripheral flange 124 between the scaling plate 136 (shown in FIGS. 7 and 8) and the crib peripheral flange 116. In some embodiments, forming the continuous peripheral seal 130 provides the stack 102 with the packing density of 0.55 g/cm³ to 0.75 g/cm³.

In some embodiments, the method 200 further includes using the plurality of vacuum channels 138 (shown in FIG. 11) located in the crib 110 in order to position the first flexible sheet 106 in the crib cavity 118. In some embodiments, the method 200 further includes bending one of the edges of the first flexible sheet 106 prior to forming the continuous peripheral seal 130 to form the first tab 132 (shown in FIG. 1). The method 200 further includes bending the corresponding edge of the second flexible sheet 128 prior to forming the continuous peripheral seal 130 to form the second tab 134 (shown in FIG. 1). As already stated above, the first tab 132 and the second tab 134 are unsealed from each other.

At step 214, the method 200 further includes removing the first flexible sheet 106 along with the stack 102 from the crib 110, as shown in FIG. 9.

Moreover, as compared to techniques for manufacturing conventional process challenge devices. the method 200 of manufacturing the process challenge device 100 of the present disclosure does not involve any manual steps of folding the first flexible sheet 106 and/or the second flexible sheet 128. Therefore, the proposed method 200 of manufacturing the process challenge device 100 may be easier to perform as compared to the conventional techniques.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A process challenge device to be used for determining the effectiveness of a sterilization procedure, the process challenge device comprising:
a stack comprising a plurality of test sheets disposed on top of each other, the stack further comprising an outer surface;
a test indicator disposed within and enclosed by the plurality of test sheets of the stack;
a first flexible sheet at least partially conforming to the outer surface of the stack, the first flexible sheet comprising a bottom wall receiving the stack thereon, a plurality of side walls extending from the bottom wall, and a peripheral flange extending from the plurality of side walls and substantially parallel to the bottom wall, wherein the bottom wall and the plurality of side walls of the first flexible sheet together define a sheet cavity therebetween, wherein the sheet cavity receives the stack therein and is dimensioned such that each of the plurality of side walls at least partially engages with the stack, wherein an inclination angle between each of the plurality of side walls and the bottom wall is from 80 degrees to 100 degrees;
a second flexible sheet disposed on and at least partially engaging with the stack and the peripheral flange of the first flexible sheet, the second flexible sheet covering the stack; and
a continuous peripheral seal coupling the second flexible sheet to the peripheral flange, such that the stack is fully enclosed by the first flexible sheet and the second flexible sheet.

2. The process challenge device of claim 1, wherein the continuous peripheral seal is a heat seal.

3. The process challenge device of claim 1, wherein the continuous peripheral seal is an ultrasonic seal.

4. The process challenge device of claim 1, wherein the first flexible sheet further comprises a first tab disposed adjacent to the continuous peripheral seal and unsealed to the second flexible sheet, and wherein the second flexible sheet comprises a second tab disposed adjacent to the continuous peripheral seal and unsealed to the first flexible sheet.

5. The process challenge device of claim 1, wherein the stack comprises a packing density from 0.55 g/cm³ to 0.75 g/cm³.

6. The process challenge device of claim 1, wherein each of the first flexible sheet and the second flexible sheet is made from a nonwoven material.

7. The process challenge device of claim 6, wherein the nonwoven material comprises a three-layered spunbond-meltblown-spunbond (SMS) construction.

8. The process challenge device of claim 6, wherein the nonwoven material comprises at least a portion of polyolefin fibers.

9. The process challenge device of claim 1, wherein each of the first flexible sheet and the second flexible sheet has a melting temperature greater than 130° C, and less than 220° C.

10. The process challenge device of claim 1, wherein each of the first flexible sheet and the second flexible sheet is permeable to a steam sterilant used in the sterilization procedure and a gas.

11. The process challenge device of claim 1, wherein each of the plurality of test sheets is made of paper, or paper and polyurethane foam.

12. The process challenge device of claim 1, wherein each of the first flexible sheet and the second flexible sheet is devoid of any openings having an area greater than or equal to 0.5 mm².

13. The process challenge device of claim 1, wherein each of the first flexible sheet and the second flexible sheet is non-thermoformable and non-moldable.

14. A method of manufacturing a process challenge device to be used for determining the effectiveness of a sterilization procedure, the method comprising:
providing a stack comprising a plurality of test sheets disposed on top of each other, the stack further comprising an outer surface;
placing a test indicator within the plurality of test sheets of the stack;
placing the stack on a first flexible sheet;
placing the first flexible sheet along with the stack within a crib comprising a crib bottom wall, a plurality of crib side walls extending from the crib bottom wall, a crib peripheral flange extending from the plurality of crib side walls and substantially parallel to the crib bottom wall, and a crib cavity defined between the crib bottom wall and the plurality of crib side walls, wherein the crib cavity at least partially receives the first flexible sheet along with the stack therein, wherein placement of the first flexible sheet along with the stack within the crib deforms the first flexible sheet to at least partially conform to the outer surface of the stack, wherein deformation of the first flexible sheet forms a bottom wall at least partially engaging with the crib bottom wall, a plurality of side walls extending from the bottom wall and at least partially engaging with the corresponding plurality of crib side walls, and a peripheral flange extending from the plurality of side walls and substantially parallel to the bottom wall, the peripheral flange of the first flexible sheet at least partially engaging with the crib peripheral flange, wherein the crib cavity is dimensioned such that:
each of the plurality of side walls of the first flexible sheet at least partially engages with the stack; and
an inclination angle between each of the plurality of side walls and the bottom wall of the first flexible sheet is from 80 degrees to 100 degrees;
placing a second flexible sheet on the stack and the first flexible sheet, such that the second flexible sheet covers the stack and at least partially engages with the stack and the peripheral flange of the first flexible sheet;
forming a continuous peripheral seal between the second flexible sheet and the peripheral flange, thereby coupling the second flexible sheet to the peripheral flange and fully enclosing the stack between the first flexible sheet and the second flexible sheet; and
removing the first flexible sheet along with the stack from the crib.

15. The method of claim 14, wherein the continuous peripheral seal is formed by heat sealing.

16. The method of claim 14, wherein the continuous peripheral seal is formed by ultrasonic sealing.

17. The method of claim 14, wherein forming the continuous peripheral seal comprises at least partially receiving the second flexible sheet and the peripheral flange between a sealing plate and the crib peripheral flange.

18. The method of claim 14, further comprising:
bending one of the edges of the first flexible sheet prior to forming the continuous peripheral seal to form a first tab; and
bending a corresponding edge of the second flexible sheet prior to forming the continuous peripheral seal to form a second tab;
wherein the first tab and the second tab are unsealed from each other.

19. The method of claim 14, further comprising using a plurality of vacuum channels located in the crib in order to position the first flexible sheet in the crib cavity.

20. The method of claim 14, wherein forming the continuous peripheral seal provides the stack with a packing density of 0.55 g/cm³ to 0.75 g/cm³.
